(19) United States
(12) Patent Application Publication
(10) Pub. No.: US 2021/0030903 A1
(43) Pub. Date: Feb. 4, 2021

(54) METHOD OF DECONTAMINATING A HYDROCARBON FLUID USING GAMMA RADIATION

(71) Applicant: Phoenix Environmental, Inc., Plymouth, MI (US)

(72) Inventors: Davis Smith, Grand Rapids, MI (US); Casey Joe Smith, Scottsdale, AZ (US)

(21) Appl. No.: 16/945,026

(22) Filed: Jul. 31, 2020

Related U.S. Application Data
(60) Provisional application No. 62/882,129, filed on Aug. 2, 2019.

(51) Int. Cl. A61L 2/08; C10G 32/04; C10G 75/00

(52) U.S. Cl. CPC A61L 2/081; C10G 32/04; C10G 75/00; C10G 2400/04; C10G 2400/02; C10G 2400/06; C10G 2300/202

(57) ABSTRACT

In an aspect, a method of decontaminating a hydrocarbon fluid comprises irradiating the hydrocarbon fluid in a storage tank with a gamma radiation to maintain or reduce an amount of a microorganism in the storage tank; wherein a source of the gamma radiation is located within and/or proximal to the storage tank. In another aspect, a method of reducing an amount of a biocorrosion comprises irradiating the biocorrosion located on a surface of a device in contact with a hydrocarbon fluid with a gamma radiation; wherein a source of the gamma radiation is located within and/or proximal to the device.

### CROSS-REFERENCE TO RELATED APPLICATION

This application claims the benefit of U.S. Provisional Patent Application Ser. No. 62/882,129 filed Aug. 2, 2019. The related application is incorporated herein in its entirety by reference.

### BACKGROUND

To protect public health and the environment, the United States Environmental Protection Agency Clean Air Highway Diesel final rule stipulated a 97% reduction in sulfur content of diesel fuel. The Tier 3 Gasoline Sulfur program that grew out of the Clean Air Act required a reduction in the sulfur content in gasoline to a maximum of 10 parts per million by weight beginning in 2017. Within one year of implementation, the Petroleum Equipment Institute received reports of severe and accelerated corrosion of metallic components of storage tanks and equipment used for transporting, dispensing, and storing ultra-low sulfur diesel. Metallic components included corrosion resistant materials such as aluminum, copper, stainless steel, galvanized steel, and more. Reports included, for example, observations of a metallic coffee ground type substance clogging the dispenser filters in addition to corrosion and/or failure of seals, gaskets, tanks, meters, leak detectors, solenoid valves, and riser pipes. Failure of such components can result in release of fuel products creating a large environmental hazard.

The presence of acetic acid or acetate in high concentration in the vapor sampled from various ultra-low sulfur diesel containing tanks, as well as the concentration of acetate in the water bottoms, suggest that acetic acid may be reacting with the iron to produce the scale and corrosion observed of the corroded equipment. As such, it is believed that the corrosion ultimately arises from increased levels of acetic acid in the ultra-low sulfur diesel, where the acetic acid is likely being produced by acetic acid producing bacteria feeding on low levels of ethanol contamination, possibly by the following reaction:

C₂H₅OH+O₂→CH₃COOH+H₂O

It was found that fuel comprising even as little as 0.0033 volume percent of ethanol in the presence of enough bacteria and oxygen could result in high enough amounts of acetic acid to cause extensive corrosion.

Acetic acid producing bacteria is likely to be the cause of the increased levels of acetic acid as bacteria of the family Acetobacteraceae was found to be present in the bottom and/or in the sediment that accumulates in, for example, the storage tanks. Bacteria of the family Acetobacteraceae, specifically of the genus Acetobacter, are known to metabolize ethanol into acetic acid in the presence of oxygen and water in slightly acidic conditions. It is believed that higher levels of acetic acid producing bacteria are present in ultra-low sulfur diesel as compared to low sulfur diesel due to the higher levels of sulfur functioning as a natural biocide in the low sulfur diesel.

Changing legislation is further compounding the issues with bacterial growth in gasoline as regulations have been encouraging, and in some cases mandating the incorporation of ethanol in gasoline. These regulations have resulted in the widespread use of E10 gasoline and the advent of E15 gasoline for flex fuel vehicles. The ample amount of the ethanol and the presence of small amounts of water inherently present in the ethanol results in large bacterial populations forming biofilms on the inner walls of the fuel containing devices such as tanks, pipes, etc., fatigue cracking in pipeline steels, and in the increased risk of explosion as the acetic acid can migrate out solution, collecting in the headspace of the storage tanks.

Current methods of controlling bacterial growth include reducing water content in fuel, decontaminating fuels using ultraviolet light, and addition of pesticides from multiple manufacturers. Fuels containing undetermined concentrations of pesticides are being used in internal combustion engines, boilers, and for other uses. The public is exposed to diesel emissions containing varying levels of combusted pesticides that have unknown health risks. Employees in the petroleum industry have a much higher potential for exposure to these pesticides.

A system and method for reducing the amount of acetic acid producing bacteria is therefore desirable to reduce the levels of acetic acid and to ultimately reduce the amount of corrosion of the equipment used in the storage and transportation of fuel.

### BRIEF SUMMARY

Disclosed herein is a method of decontaminating a hydrocarbon fluid using gamma radiation.

In an aspect, a method of decontaminating a hydrocarbon fluid comprises irradiating the hydrocarbon fluid in a storage tank with a gamma radiation to maintain or reduce an amount of a microorganism in the storage tank; wherein a source of the gamma radiation is located within and/or proximal to the storage tank.

In another aspect, a method of reducing an amount of a biocorrosion comprises irradiating the biocorrosion located on a surface of a device in contact with a hydrocarbon fluid with a gamma radiation; wherein a source of the gamma radiation is located within and/or proximal to the device.

The above described and other features are exemplified by the following figures, detailed description, and claims.

### BRIEF DESCRIPTION OF THE DRAWINGS

The figures are exemplary embodiments, wherein the like elements are numbered alike. The figures are provided to illustrate the present disclosure and are not intended to limit devices made in accordance with the disclosure to the materials, conditions, or process parameters set forth herein.

FIG. 1 is an illustration of a storage tank; and

FIG. 2 is also an illustration of a storage tank.

### DETAILED DESCRIPTION

Recent regulatory changes with regards to hydrocarbon fluids such as diesel and gasoline have mandated a reduction in the sulfur concentration. Government mandates have encouraged increases of ethanol concentrations in gasoline blends. These changes are believed to be the cause of significant increases in the levels of corrosion arising from increased populations of microorganisms that can convert the ethanol to acetic acid. To inhibit the increased growth of microorganism and biocorrosion occurring in fuel systems, a method of decontaminating the hydrocarbon fluid using gamma radiation was developed. The present method has the benefit of an increased decontamination ability, for example, as compared to decontamination with an ultraviolet light source. Decontamination with gamma radiation has additional advantages in that the gamma radiation source can be located directly in a storage tank, avoiding a need for an external decontamination unit and that it can effectively decontaminate the headspace region of the storage tank that is not in direct contact with the hydrocarbon fluid.

Specifically, the method of decontaminating a hydrocarbon fluid can include irradiating the hydrocarbon fluid in a storage tank with gamma radiation to maintain or reduce an amount of a microorganism in the storage tank. The source of the gamma radiation can be located within the storage tank. For example, the storage tank can be opened, the source of the gamma radiation can be inserted, and the storage tank can be closed. In order to increase the exposure of the hydrocarbon fluid to the gamma radiation, the storage tank can comprise a mixing element such as a rotating shaft, a magnetic stirrer, or a pump (for example, internally or externally located with respect to the storage tank).

The method of decontaminating the hydrocarbon fluid can comprise flowing a contaminated hydrocarbon fluid into a decontamination unit, irradiating the hydrocarbon fluid with gamma radiation that is emitted from a gamma radiation emitting source such that the contaminated hydrocarbon fluid becomes a more purified hydrocarbon fluid, and flowing the purified hydrocarbon fluid out of the decontamination unit; wherein a microorganism level in the purified hydrocarbon fluid is less than that of the contaminated hydrocarbon fluid.

If the hydrocarbon fluid is located in a hydrocarbon fluid transportation unit, then the method of decontamination of hydrocarbon fluid in the transportation unit can comprise irradiating a decontamination region of the hydrocarbon fluid transportation unit with a gamma radiation emitting source configured to irradiate the hydrocarbon fluid with gamma radiation. The decontamination region can be in the hydrocarbon fluid transportation unit or can be a separation region from the hydrocarbon fluid transportation unit.

The irradiation dosage can be constant or intermittent. Constant dosage can be applied using low level sources, which can emit gamma rays as low as 1 kilo-Gray (kGy). Intermittent dosage can use higher levels of greater than or equal to 10 kGy. As used herein intermittently irradiating can comprise irradiating for a first amount of time to reduce a microorganism level to below a predetermined level; after achieving the predetermined level, stopping the irradiating for a second amount of time until a maximum microorganism level is achieved; and after achieving the maximum microorganism level, irradiating the hydrocarbon fluid. The irradiation dosage can be applied for time intervals of 0.1 second to 1 week.

The source of the gamma radiation can be positioned such that the gamma radiation reaches an inner surface of the storage tank to reduce the amount of or to prevent the occurrence biocorrosion on the inner surface. The source of the gamma radiation can be positioned such that the gamma radiation reaches greater than or equal to 50 area percent, or 75 to 100 area percent, or 95 to 100 area percent of the inner surface of the storage tank. For large storage tanks, multiple sources of the gamma radiation can be present in and/or proximal to the storage tank such that all of the inner surface of the storage tank is exposed to the gamma radiation. The gamma radiation can prevent the build-up of biocorrosion on the inner surface of the storage tank.

The source of the gamma radiation can be positioned such that the gamma radiation irradiates a head space of the storage tank, which can result in a reduction in the amount of biocorrosion in the head space or can prevent the biocorrosion in the head space from occurring. The source of the gamma radiation can be located in the head space of the storage tank. As used herein, the head space refers to a gas filled volume in the storage tank.

The source of the gamma radiation can be located outside of the storage tank and proximal to a wall of the storage tank, provided the walls of the storage tank are permeable to the gamma radiation. For example, the source of the gamma radiation can be located within 5 centimeters from an outer surface of the storage tank.

A hydrocarbon fluid decontamination unit can comprise a decontamination region containing hydrocarbon fluid and a gamma radiation emitting source located in the decontamination region that can be configured to irradiate the hydrocarbon fluid.

The method has the added benefit that the storage tank can be located underground. For example, at least 10 volume percent, or 10 to 100 volume percent of the storage tank can be located underground. When 100 volume percent of the storage tank is located underground, the separating material (for example, at least one of earth, concrete, brick, steel, or the like) can reduce or prevent the gamma radiation from reaching a ground-level surface. In other words, a person standing on the ground on top of the storage tank can have an extremely low to no risk of being exposed to the gamma radiation originating from the underground storage tank.

The walls of the storage tank can comprise at least one of fiber glass or steel. If the storage tank comprises fiber glass, then the storage tank further can comprise an outer shielding layer; wherein the outer shielding layer optionally comprises at least one of lead or steel. The walls of the storage tank and/or the shielding layer can be configured such that the gamma radiation can be prevented from exiting the storage tank, thereby reducing the risk of or preventing the exposure of a by-stander of being exposed to gamma radiation originating from the source of the gamma radiation. A wall thickness of the storage tank and the shielding layer can each independently be 1 to 10 centimeters, or 2 to 5 centimeters.

The storage tank can have an inner volume of greater than or equal to 20 meters cubed, or 30 to 100 meters cubed. The storage tank can be capable of storing 55 to 160,000 liters, or 150,000 to 155,000 liters of the hydrocarbon fluid.

The hydrocarbon fluid can be filtered to remove any particulates or contaminant present in hydrocarbon fluid, for example, arising from irradiated microorganisms. The hydrocarbon fluid can be filtered by directing a stream of particulate-containing hydrocarbon fluid through a filter to form a filtered stream. The filtered stream can be redirected to the storage tank. The filter can have a pore size of less than or equal to 100 micrometers, or less than or equal to 50 micrometers, or 5 to 10 micrometers.

The hydrocarbon fluid can comprise at least one of petroleum, gasoline (for example, E10, E15, or E85), heating oil, diesel fuel (for example, biodiesel), kerosene, or jet fuel.

The source of the gamma radiation can comprise at least one of cobalt-60, strontium-90, cesium-137 and barium-137, or iridium-192.

The microorganism can comprise at least one of a bacterium or a fungi. An example of a microorganism that utilizes hydrocarbons and can be present in the hydrocarbon fluid is Pseudomonas aeruginosa. Other types of microorganisms include bacteria such as Desulfovibrio desulfuricans, Flavobacterium species, Micrococcus paraffivae, Mycobacterium phlei, Bacterium aliphaticum, etc., and fungi such as Cladosporium, Nocardia, Aspergillus, Candida lipolytica, Penicillium, etc. The microorganism can comprise a lactic acid bacterium capable of converting an alcohol, such as ethanol, to an organic acid, such as lactic acid or acetic acid. Examples of lactic acid bacteria include those in the Lactobacillus species, those in the Pediococcus species, Acetobacter species, or wild yeast.

Treatment of hydrocarbon fluid can kill greater than or equal to 90 weight percent of the microorganisms in the contaminated hydrocarbon fluid, or greater than or equal to 95 weight percent, or 99 to 99.99 weight percent based on the total weight of the microorganisms present prior to exposure to the gamma radiation.

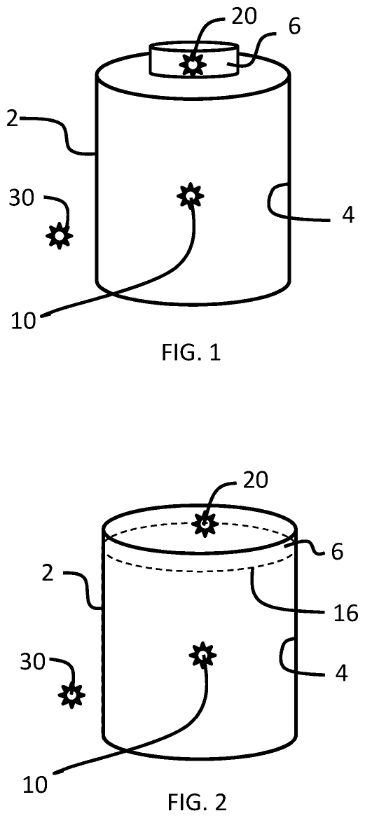

FIG. 1 and FIG. 2 are illustrations of non-limiting aspects of storage tank 2. Storage tank 2 has an inner surface 4 and a head space 6 as defined by a region above the liquid level 16, where the liquid level 16 is not illustrated in FIG. 1. The figures illustrate that an internal gamma source 10 can be located in the storage tank 2, a headspace gamma source 20 can be located in the headspace 6, and/or an external gamma source 30 can be located proximal to the storage tank 2.

Set forth below are various non-limiting aspects of the present disclosure.

Aspect 1: A method of decontaminating a hydrocarbon fluid comprising: irradiating the hydrocarbon fluid in a storage tank with a gamma radiation to maintain or reduce an amount of a microorganism in the storage tank; wherein a source of the gamma radiation is located within and/or proximal to the storage tank.

Aspect 2: The method of Aspect 1, further comprising mixing the hydrocarbon fluid in the storage tank.

Aspect 3: The method of any one or more of the preceding aspects, wherein the irradiating comprises irradiating an inner surface of the storage tank to reduce the amount of biocorrosion on the inner surface or to prevent the biocorrosion from forming on the inner surface.

Aspect 4: The method of any one or more of the preceding aspects, wherein the irradiating comprises irradiating a head space of the storage tank to reduce the amount of biocorrosion in the head space or to prevent the biocorrosion in the head space.

Aspect 5: The method of any one or more of the preceding aspects, wherein at least 10 volume percent, or 10 to 100 volume percent of the storage tank is located underground.

Aspect 6: The method of Aspect 5, wherein 100 volume percent of the storage tank is located underground such that the gamma radiation does not reach a ground-level surface.

Aspect 7: The method of any one or more of the preceding aspects, wherein the storage tank has an inner volume of greater than or equal to 20 meters cubed, or 30 to 100 meters cubed; and/or wherein the storage tank can hold 55 to 160,000 liters, or 150,000 to 155,000 liters of the hydrocarbon fluid.

Aspect 8: The method of any one or more of the preceding aspects, wherein the storage tank comprises at least one of fiber glass or steel.

Aspect 9: The method of Aspect 8, wherein the storage tank comprises fiber glass and the storage tank further comprises an outer shielding layer; wherein the outer shielding layer optionally comprises at least one of lead or steel.

Aspect 10: The method of any one or more of the preceding aspects, further comprising filtering the hydrocarbon fluid.

Aspect 11: A method of reducing the amount of a biocorrosion comprising: irradiating the biocorrosion located on a surface of a device in contact with a hydrocarbon fluid with a gamma radiation; wherein a source of the gamma radiation is located within and/or proximal to the device.

Aspect 12: The method of Aspect 11, wherein the device is a storage tank, a pump, a filter, or a pipeline.

Aspect 13: The method of any one or more of the preceding aspects, wherein the irradiating with the gamma radiation is constant.

Aspect 14: The method of any one or more of Aspects 1 to 12, wherein the irradiating with the gamma radiation comprises intermittently irradiating with the gamma radiation.

Aspect 15: The method of Aspect 14, wherein the intermittently irradiating comprises irradiating for a first amount of time to reduce a microorganism level to below a predetermined level; after achieving the predetermined level, stopping the irradiating for a second amount of time until a maximum microorganism level is achieved; and after achieving the maximum microorganism level, irradiating the hydrocarbon fluid.

Aspect 16: The method of any one or more of the preceding aspects, wherein the hydrocarbon fluid comprises at least one of petroleum, gasoline (for example, E10, E15, or E85), heating oil, diesel fuel (for example, biodiesel).

Aspect 17: The method of any one or more of the preceding aspects, wherein the hydrocarbon fluid comprises less than or equal to 15 parts per million by weight of sulfur.

Aspect 18: The method of any one or more of the preceding aspects, wherein the hydrocarbon fluid comprises greater than or equal to 10 volume percent of ethanol based on the total volume of the hydrocarbon fluid.

Aspect 19: The method of any one or more of the preceding aspects, wherein the source of the gamma radiation comprises at least one of cobalt or cesium.

Aspect 20: The method of any one or more of the preceding aspects, wherein the microorganism comprises at least one of a bacteria of the family Acetobacteraceae, a bacteria of the family Lactobacillaceae, or a fungi of the family Saccharomycetaceae.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

The terms “a” and “an” do not denote a limitation of quantity, but rather denote the presence of at least one of the referenced item. The term “or” means “and/or” unless clearly indicated otherwise by context. Reference throughout the specification to “an aspect”, “an embodiment”, “another embodiment”, “some embodiments”, and so forth, means that a particular element (e.g., feature, structure, step, or characteristic) described in connection with the embodiment is included in at least one embodiment described herein, and may or may not be present in other embodiments. In addition, it is to be understood that the described elements may be combined in any suitable manner in the various embodiments.

The endpoints of all ranges directed to the same component or property are inclusive of the endpoints, are independently combinable, and include all intermediate points and ranges. For example, ranges of “up to 25 volume percent, or 5 to 20 volume percent” is inclusive of the endpoints and all intermediate values of the ranges of “5 to 25 volume percent,” such as 10 to 23 volume percent, etc.).

The term “combination” is inclusive of blends, mixtures, alloys, reaction products, and the like. Also, “combinations comprising at least one of the foregoing” means that the list is inclusive of each element individually, as well as combinations of two or more elements of the list, and combinations of at least one element of the list with like elements not named.

Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method of decontaminating a hydrocarbon fluid comprising:
irradiating the hydrocarbon fluid in a storage tank with a gamma radiation to maintain or reduce an amount of a microorganism in the storage tank;
wherein a source of the gamma radiation is located within and/or proximal to the storage tank.

2. The method of claim 1, further comprising mixing the hydrocarbon fluid in the storage tank.

3. The method of claim 1, wherein the irradiating comprises irradiating an inner surface of the storage tank to reduce the amount of biocorrosion on the inner surface or to prevent the biocorrosion from forming on the inner surface.

4. The method of claim 1, wherein the irradiating comprises irradiating a head space of the storage tank to reduce the amount of biocorrosion in the head space or to prevent the biocorrosion in the head space.

5. The method of claim 1, wherein at least 10 volume percent of the storage tank is located underground.

6. The method of claim 5, wherein 100 volume percent of the storage tank is located underground such that the gamma radiation does not reach a ground-level surface.

7. The method of claim 1, wherein the storage tank has an inner volume of greater than or equal to 20 meters cubed; and/or wherein the storage tank can hold 55 to 160,000 liters of the hydrocarbon fluid.

8. The method of claim 1, wherein the storage tank comprises at least one of fiber glass or steel; wherein if the storage tank comprises fiber glass then the storage tank further comprises an outer shielding layer that comprises at least one of lead or steel.

9. The method of claim 1, further comprising filtering the hydrocarbon fluid.

10. The method of claim 1, wherein the irradiating with the gamma radiation comprises intermittently irradiating with the gamma radiation.

11. The method of claim 10, wherein the intermittently irradiating comprises irradiating for a first amount of time to reduce a microorganism level to below a predetermined level; after achieving the predetermined level, stopping the irradiating for a second amount of time until a maximum microorganism level is achieved; and after achieving the maximum microorganism level, irradiating the hydrocarbon fluid.

12. The method of claim 1, wherein the hydrocarbon fluid comprises at least one of petroleum, gasoline, heating oil, or diesel fuel.

13. The method of claim 1, wherein the hydrocarbon fluid comprises less than or equal to 15 parts per million by weight of sulfur.

14. The method of claim 1, wherein the hydrocarbon fluid comprises greater than or equal to 10 volume percent of ethanol based on the total volume of the hydrocarbon fluid.

15. The method of claim 1, wherein the source of the gamma radiation comprises at least one of cobalt or cesium.

16. The method of claim 1, wherein the microorganism comprises at least one of a bacteria of the family Acetobacteraceae, a bacteria of the family Lactobacillaceae, or a fungi of the family Saccharomycetaceae.

17. A method of reducing the amount of a biocorrosion comprising:
irradiating the biocorrosion located on a surface of a device in contact with a hydrocarbon fluid with a gamma radiation;
wherein a source of the gamma radiation is located within and/or proximal to the device.

18. The method of claim 17, wherein the device is a storage tank, a pump, a filter, or a pipeline.

19. The method of claim 17, wherein the irradiating with the gamma radiation is constant.

20. A method of decontaminating a hydrocarbon fluid comprising:
irradiating the hydrocarbon fluid in a storage tank with a gamma radiation to maintain or reduce an amount of at least one of a bacteria of the family Acetobacteraceae, a bacteria of the family Lactobacillaceae, or a fungi of the family Saccharomycetaceae in the storage tank; and
filtering the hydrocarbon fluid;
wherein the hydrocarbon fluid comprises at least one of petroleum, gasoline, heating oil, or diesel fuel; wherein the hydrocarbon fluid comprises less than or equal to 15 parts per million by weight of sulfur and greater than or equal to 10 volume percent of ethanol based on the total volume of the hydrocarbon fluid.
